(19) United States
(12) Patent Application Publication
(10) Pub. No.: US 2004/0171916 A1
(43) Pub. Date: Sep. 2, 2004

(54) Endoscope with a lockable optic system

(76) Inventor: Pieter Brommersma, Bargteheide (DE)

Correspondence Address: RANKIN, HILL, PORTER & CLARK, LLP, 925 EUCLID AVENUE, SUITE 700, CLEVELAND, OH, 44115-1405, US

(21) Appl. No.: 10/483,108

(22) PCT Filed: Jun. 17, 2002
(86) PCT No.: PCT/EP02/06646

(30) Foreign Application Priority Data
Aug. 10, 2001 (DE) 101 39 444.6

(51) Int. Cl. A61B 1/06

(52) U.S. Cl. 600/162

(57) ABSTRACT

The invention relates to a medical endoscope (1) that comprises a proximal end piece (3) and an optical system (4) that can be inserted up to a proximal eyepiece (5). A spring-loaded locking device is configured in the end piece and engages in a groove (8) of a pin (7) in a locking manner. Said pin is provided with a tip (9), distally projects from the eyepiece and can be inserted in the groove. A slide (12a) is disposed in a shaft (11) that extends in the end piece (3) and can be actuated from the shaft end. Said slide is coupled to one or more blades (15a) in such a manner that it lifts them off the pin when the device is actuated, the blades being slideable cross-wise to the pin in the end piece and being pressed against the pin under the action of a spring.

### BACKGROUND OF THE INVENTION

1. Field of the Invention

The present invention relates to a medical endoscope having a proximal terminal segment and an optics that may be inserted as far as a proximal ocular segment, and having a spring loaded locking system.

2. Description of Related Art

Besides other devices, for instance surgical implements, rinsing ducts, and the like, medical and, in particular, urological endoscopes such as resectoscopes contain within an elongated duct an essential component, namely an optics. Conventionally, such optics consist of an elongated tube housing optical elements such as lens elements of fiber optics guides as well as any light guides used for illumination. An ocular device comprising an ocular, a camera, or a camera adapter is mounted on the proximal end.

The optics may be retracted or withdrawn from endoscopes of this kind, allowing easier cleaning and, furthermore, allowing the exchange of one optics for another with a different direction of viewing.

In order to secure the optics against accidental displacement of its operational position, the endoscope is fitted with a locking system at a terminal segment of the endoscope. The locking system itself is locked in position by a pin distally projecting from the optics' ocular device. The pin is fitted with a channel that may be elastically engaged by the locking system.

In known endoscopes of the above kind, the locking system comprises one or several spring-loaded balls that snap into the channel. Easy penetration of the pin between the balls is made possible by the pin's tip. Illustratively, a device of the above design is disclosed in the German patent document 39 17 583 Al.

This known design assures easy engagement but also similarly-easy disengagement. While easy engagement is advantageous when assembling the endoscope, easy disengagement, on the other hand, is a drawback.

Substantial problems were not encountered in earlier endoscopes fitted with a single ocular at the end of the optics even in the event of an easily disengaging locking system. However, difficulties will be encountered as regards the video cameras mounted to the ocular device of the later technology. If during surgery an auxiliary person should step on the camera cable, the easily disengaging optics will be pulled out of the endoscope. In the midst of critical surgery, the surgeon then no longer will see a display on the monitor.

The German patent document 19 71 5510 C2 describes a coupling outside the above state of the art between the endoscope optics and a camera, and, during the action of locking, a spring-loaded latch snapping into a channel configured externally in the optics' terminal segment. This latch is manually operable against the spring force in order to detach the camera module from the endoscope.

### SUMMARY OF THE INVENTION

An object of the present invention is to design a medical endoscope of the above-cited kind in which accidental detachment of the optics is precluded.

In accordance with the present invention, the pin's channel is entered not by a ball or another rounded element easily disengaged by traction from the pin, but rather by a blade or blade element which engages in a mechanically interlocking manner the channel the way blocking extensions or sliders do in order to assure full reliability against being pulled out. The blade or blade element shall be raised only intentionally by means of the slider so as to release the pin. Illustratively, the blade element may engage by means of a proximal surface orthogonal to the pin's axis a proximal channel surface that is orthogonal to the pin axis. Snapping into position may be implemented in simple and conventional manner in that the tip at the pin's end shall elastically push aside the blade element.

If only one slider is being driven, unintended actuations may occur if the surgeon during a critical situation would act somewhere on the endoscope, namely accidentally on the slider and thereby would unlock the optics. However, in accordance with the present invention, two or more sliders shall be actuated simultaneously, which shall only then raise one or more blade elements entering the channel and thereby provide additional reliability against accidental actuation.

Advantageously the linkage of the slider(s) to one or more blade elements shall be carried out in a manner that offers the advantage of simplified actuation by one-finger pressure compared with the alternative of slider extraction entailing cumbersome use of several fingers.

In further accordance with the present invention, two mutually opposite sliders that are actuated at the terminal segment and may be operated in very simple manner with mutually opposite thumb and index finger.

A slider is linked, by means of reversing levers or the like, to a blade element such that, when said slider is depressed, the blade element shall lift off, and locking illustratively is implemented when the slider and the blade element are configured on the same side of the pin. However, in further accordance with the present invention, the slider by means of its extension supports the blade element positioned on the other pin side, the blade element enters the pin's channel from this position, that is “from the rear”. In this manner and without recourse to in-line gear elements, when pressing the slider, the blade element may be lifted off the pin by being moved in the same direction. This design can be implemented by very simple assembly and most importantly in this instance, in a most compact manner.

Furthermore, the above design may be most advantageously configured in the form of one slider on each side of the pin. The two sliders may be actuated by pressure being applied from mutually opposite sides of the terminal segment to attain disengagement.

In further accordance with the present invention, a compression spring may be mounted between the blade element and the slider on each side, one spring sufficing to elastically load both sliders and blade elements.

### BRIEF DESCRIPTION OF THE DRAWINGS

The invention is elucidated in illustrative and schematic manner in the appended drawings, wherein:

FIG. 1 is a top view of the proximal end zone of an optics-equipped endoscope of the present invention,

FIG. 2 is a side view relating to FIG. 2,

FIG. 3 is an enlarged section along the line 3-3 in FIG. 2 of the locking system of the invention,

FIG. 4 is a second embodiment of the said locking system shown in section as in FIG. 3, and

FIG. 5 shows the slider of FIG. 4 seen in the endoscope's axial direction.

### DETAILED DESCRIPTION OF THE PREFERRED EMBODIMENTS

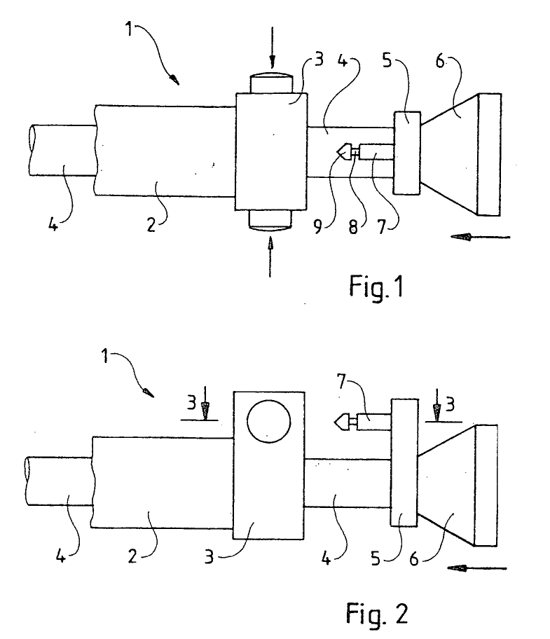

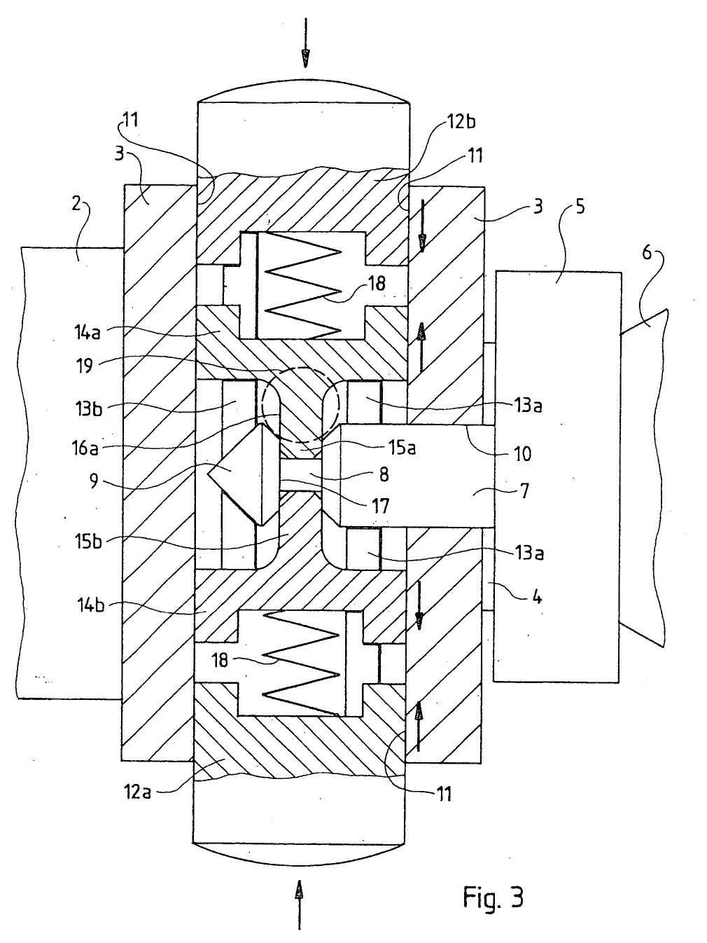

FIGS. 1-3 show a first embodiment of the present invention, the cross-section of FIG. 3 being considerably enlarged to show the details.

The drawings show in highly diagrammatic manner an endoscope 1 comprising a hollow stem 2 and a proximal terminal segment 3. An optics 4 may be inserted through the terminal segment 3 and bears, at its proximal end, an ocular segment 5 with an ocular 6. In FIGS. 1 and 2, the optics 4 is shown situated shortly before it is fully inserted, the ocular segment 5 being still some distance away from the proximal terminal segment 3. A pin 7, which is fitted with an annular channel 8 and a tip 9, projects distally from the ocular segment 5 toward the proximal terminal segment 3. Upon further insertion of the optics 4 in the direction of the arrow shown in FIGS. 1 and 2, each near the ocular 6, the pin 7 moves inside a borehole 10 shown in FIG. 3 as far as the terminal position shown in this FIG. 3.

A duct or passageway 11 crossing the proximal terminal segment 3 is configured transversely to the pin 7 and houses displaceably supported sliders 12a, 12b. Each of the sliders project outwardly from the proximal terminal segment 3 from mutually opposite ends of the duct or passageway 11. As shown in FIG. 3, the slider 12a is represented being underneath the pin 7 and the slider 12b is shown above it. The different notations 12a and 12b are merely descriptive. The sliders may be identical.

At its inside surface facing the other slider 12b, the slider 12a comprises an extension 13a in the form of an elongated arm running laterally past the pin 7 and, in FIG. 3, underneath the pin 7 as far as its opposite side where the extension 13a supports a blade element support 14a.

Relative to the pin 7, the blade-element support 14a is situated opposite the associated slider 12a. The blade-element support 14a supports at its side facing the pin 7 a blade element 15a, which, as shown in FIG. 3, enters the channel 8 of the pin 7 in a geometrically locking manner. In particular, by having the proximal end face of the blade element 15a situated perpendicularly to an axis of the pin 7, the blade element 15a rests against a proximal surface 17 of the pin 7 that is also perpendicular to the pin axis.

In identical manner, the slider 12b is connected by an extension 13b with a blade-element support 14b supporting a blade element 15b.

It must be kept in mind that the extensions 13a and 13b are required not only to run freely and shall laterally pass near the pin 7, but furthermore that they must pass around the particular blade-element support 14b and 14a, resp., of the other slider 12b and 12a, the sliders for that purpose being optionally fitted with omitted clearances.

For purposes of clarity, FIG. 3 shows a design whereby the two extensions 13a and 13b are configured on the same side of the pin 7 (underneath it in FIG. 3). Advantageously however and even as regards the design of FIG. 3, illustratively one extension may be configured to the right and underneath the said pin and the other pin left and above it. In such a case the cooperating parts of both sliders 12a and 12b may be fully identical.

In each case and on both sides, a helical spring 18 is mounted between the sliders 12b, 12a and the associated blade-element supports 14a, 14b. Nevertheless, it would be enough if only one spring were configured on one side. As a result the sliders 12a and 12b are forced apart and assure that the blade elements 15a and 15b engage the channel 8 of the pin 7.

If the optics 4 together with the pin 7 is moved farther distally in the direction of the arrows of FIGS. 1 and 2 then, as shown in FIG. 3, the tip 9 of the pin 7 will be moved between the blade elements 15a and 15b, which are appropriately fitted with slight bevels at their inner edges. The blade elements 15a, 15b shall be forced apart against their spring loading and the pin 7 shall advance farther until it snaps into the position of FIG. 3.

An optics 4 comprising a pin 7 with channel 8, such as shown in FIGS. 1-3, also may be employed with a conventional endoscope fitted with ball detent means as indicated in FIG. 3. A ball 19 is shown in dashed line in FIG. 3 and would be seated in an appropriate site in a locking system of the state of the art and being spring-loaded, it would rest against the edge of the channel 8. FIG. 3 also shows that by retracting the pin 7, the ball 19 shall be easily disengaged and release the pin.

The locking system shown in FIG. 3 is disengaged by actuating the two sliders 12a and 12b using two fingers of one hand which seize and compress in the direction of the arrows, the terminal slider surfaces rounded for this purpose. The two blade elements 15a and 15b are simultaneously lifted out of the channel 8 and thereby release the pin 7 and hence the optics 4.

The embodiment shown in FIG. 3 also may comprise only one slider, for instance the slider 12a fitted with a pertinent blade-element support 14a. In this design the slider 12a or the blade-element support 14a must be supported in another resilient manner. Illustratively, a spring might act on the terminal surface of the blade-element support 14a away from the slider 12a, with the spring resting against the end of the duct that would be closed at the site shown occupied in FIG. 3 by the slider 12b.

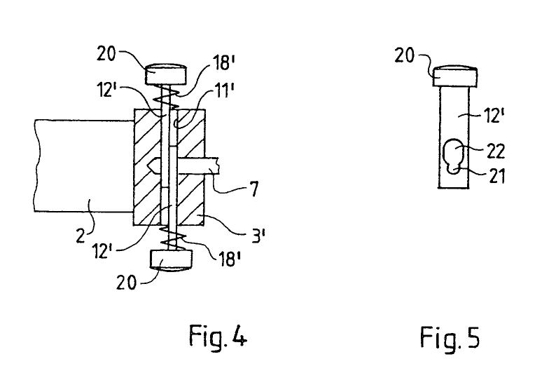

FIGS. 4 and 5 show another, similar and much simplified embodiment of the locking system of FIG. 3. These FIGS. 4 and 5 merely indicate the stem tube 2 and the proximal terminal segment 3′. The optics is omitted, though the pin 7 is shown in its detent position. This pin 7 may exhibit the same channel and tip as are shown in FIG. 3.

A flat duct 11′ crosses the proximal terminal segment 3′ transversely to the pin 7 and it receives two elongated, identical plate sliders 12′ inserted in it while resting against each other. Each slider 12′ is fitted at its external end with a drive knob 20 resting by the loading from a spring 18′ against the terminal segment 3′.

Each slider comprises an elongated slot fitted with a narrower end zone 21 and a wider end zone 22. The thickness of each slider 12′ is less than half the width of the channel 8 of the pin 7.

As shown in FIG. 4, the two sliders 12′ enter the channel 8 of the pin 7 by means of the elongated slot's narrower end zone 21 and thereby secure said pin. Only when both sliders 12′ are pressed inward due to the actuation of the-two knobs 20 shall the widened elongated slot end zones 22 in the sliders 12′ arrive in the zone of the pin 7 which thereby may be pulled out. Even a design with only one of the two sliders 12′ operates satisfactorily. Such a design merely lacks the redundant reliability against accidental release, which is assured when using two jointly operated sliders.

1. A medical endoscope (1) comprising a proximal terminal segment (3) and an optics (4) which may be inserted as far as a proximal ocular segment (5), further comprising a spring-loaded locking system configured in the terminal segment for the purpose of locking into a channel (8) of a pin (7) which is fitted with a tip (9) and which projects distally from the ocular segment, characterized in that
a slider (12a) is supported in a duct (11) extending in the terminal segment (3) and may be actuated from an end of said duct, said slider (12a) being linked in such manner with one or several blade elements (15a) which are displaceably supported in the terminal segment transversely to the pin and resiliently rest against the pin in such manner that said slider upon being actuated shall lift said blade elements (15a) off the pin.

2. Endoscope as claimed in claim 1, characterized in that it includes a second slider (12b) which may be actuated through the mouth of a second duct and is linked in such manner to one of the blade elements (15b) that all blade elements (15a, 15b) shall be lifted only if the two sliders (12a, 12b) are actuated simultaneously.

3. Endoscope as claimed in claim 1, characterized in that the linkage is designed in a manner that the minimum of one blade element (15a, 15b) shall detach when pressing the slider (12a, 12b) into the duct (11).

4. Endoscope as claimed in claim 2, characterized in that two duct mouths are situated one each on opposite sides of the terminal segment (3).

5. Endoscope as claimed in claim 3, characterized in that the duct (11) runs as far as beyond the pin (7) and in that the slider (12a, 12b) is fitted with an extension (13a, 13b) which projects from said slider's inside end into the duct while laterally enclosing the said pin and includes a blade element (15a, 15b) which engages from there.

6. Endoscope as claimed in claims 2, 4 and 5, characterized in that the duct (11) crosses the terminal segment (3) and receives a slider (12a, 12b) on each side of the pin (7).

7. Endoscope as claimed in claim 6, characterized in that a compression spring (18) is mounted at least on one side of the pin (7) between a slider (12a, 12b) and the blade element (15b, 15a) of the other slider (12b, 12a).
